(19) United States
(12) Patent Application Publication
(10) Pub. No.: US 2024/0061163 A1
(43) Pub. Date: Feb. 22, 2024

(54) THERAPUETIC WEARABLE WITH RED AND INFRARED LIGHT

(71) Applicant: Biothread LLC, Wayne, PA (US)

(72) Inventors: Jay Tapper, Wayne, PA (US); Daniel Bish, New York, NY (US); Daniel Shuter, New York, NY (US); Kristien Del Ferraro, Stamford, CT (US)

(21) Appl. No.: 17/892,302

(22) Filed: Aug. 22, 2022

(51) Int. Cl. F21V 8/00; A61N 5/06

(52) U.S. Cl. CPC G02B 6/001; A61N 5/0613; A61N 2005/0645; A61N 2005/063

(57) ABSTRACT

A light therapy wearable includes a fabric panel, at least two side-emitting optical fibers, a red light source and an infrared (IR) light source. The at least two side-emitting optical fibers are affixed to or held against the fabric panel. Each of the side-emitting optical fibers includes a core being made from a synthetic polymeric material. The red light source is configured to emit light having a wavelength of about 660 nm and is optically connected with a red light side-emitting optical fiber of the at least two side-emitting optical fibers. The IR light source is configured to emit light having a wavelength of about 830 nm and less than 850 nm. The IR light source is optically connected with an IR light side-emitting optical fiber of the at least two side-emitting optical fibers.

### BACKGROUND

Clinical studies have demonstrated the ergogenic and prophylactic benefits of red and infrared light therapy. Red and infrared light has been found to increase blood flow to the muscles and joints, which creates an anti-inflammatory response, in addition to providing increased pliability. Muscle and joint stiffness and soreness have been demonstrated to be significantly reduced while muscle contractile function was simultaneously improved by using red and infrared light therapy.

Red and infrared (IR) light therapy is utilized by professional athletes, in addition to being widely available in medical spas and physical therapy outlets. Likewise, at the very high end of the consumer market, light therapy awareness and usage has grown. Building on this awareness, there has also been a plethora of non-light-based wraps and sleeves that have invaded social media, espousing anti-inflammatory and muscle recovery messages. While affordable and accessible, these products only provide temporary relief of symptoms, with little to no recovery or preventative benefits.

Clothing made from light emitting fabrics is described in U.S. Pat. No. 4,234,907. This patent, however, describes such clothing as a fad item or as safety clothing to emit light outward when the wearer wishes to be seen by others. US 2013/0116612 A1 discloses an illuminatable pad in FIG. 4 and different types of braces or supports in FIGS. 10-13. The pads, braces and supports disclosed in US 2013/0116612 A1 are described as including elongated polymeric tubes sold by Poly Optics Australia, Pty Ltd, which according to their literature have a gel core made from optically pure case acrylic monomers with a smallest available outer diameter (OD) for the core being 3.0 mm. Poly Optics Australia, Pty Ltd also sells the core placed in a jacket, which is described as a “Poly Jacket,” and the smallest available OD when provided in the jacket according to the available literature is 5.0 mm.

There are problems that need to be overcome to provide a light therapy wearable that incorporates side-emitting optical fibers to distribute red and IR light to a wearer. These problems are associated with the attenuation of the red and IR light as it travels through the side-emitting optical fibers.

### SUMMARY

In view of the foregoing, a light therapy wearable includes a fabric panel, at least two side-emitting optical fibers, a red light source and an infrared (IR) light source. The at least two side-emitting optical fibers are affixed to or held against the fabric panel. The red light source is configured to emit light having a wavelength between 630 and 700 nm and is optically connected with a red light side-emitting optical fiber of the at least two side-emitting optical fibers. The IR light source is configured to emit IR light having a wavelength of less than 850 nm. The IR light source is optically connected with an IR light side-emitting optical fiber of the at least two side-emitting optical fibers and includes a core being made from a synthetic polymeric material.

For the light therapy wearable in the above paragraph, the wavelength of light from the IR light source has a spectral attenuation in the IR light side-emitting optical fiber no more than 7 times that of a spectral attenuation of light from the red light source in the red light side-emitting optical fiber. Additionally, the red light source can be a first red light source, and the light therapy wearable can further include a second red light source, and the first red light source can project light into a first end of the red light side-emitting optical fiber and the second red light source can project light into a second end of the red light side-emitting optical fiber, where the second end is opposite the first end. Additionally, the fabric panel can be a first fabric panel, and the light therapy wearable can further include a second fabric panel sewn or otherwise affixed to the first fabric panel, where the first red light source is mounted to the first fabric panel and the second red light source is mounted to the second fabric panel. Additionally, the IR light side-emitting optical fiber can be a first IR light side-emitting optical fiber, and the light therapy wearable can further include a second IR light side-emitting optical fiber, where the first IR light side-emitting optical fiber is affixed to or held against the first fabric panel and the second IR light side-emitting optical fiber is affixed to or held against the second fabric panel. Additionally, the IR light source can be a first IR light source, and the light therapy wearable can further include a second IR light source, where the first IR light source is mounted to the first fabric panel and the second IR light source is mounted to the second fabric panel. Additionally, the first IR light side-emitting optical fiber can have two ends and the first IR light source projects light into each end of the two ends of the first IR light side-emitting optical fiber, and the second IR light side-emitting optical fiber can have two ends and the second IR light source projects light into each end of the two ends of the second IR light side-emitting optical fiber. Additionally, the light therapy wearable can further include a first light pod including a first light pod housing mountable to the first fabric panel and a second light pod including a second light pod housing mountable to the second fabric panel, where the first red light source and the first IR light source are disposed in the first light pod housing and the second red light source and the second IR light source are disposed in the second light pod housing. Additionally, the light therapy wearable can further include a red light side-emitting light tube including the red light side-emitting optical fiber, which includes a core, and a red light jacket at least partially surrounding the red light side-emitting optical fiber, where the red light jacket is made from a light-transmissive material and having a durometer of at least 50 A and an outer diameter at least seven times a core diameter of the red light side-emitting optical fiber. Additionally, the light therapy wearable can further include an IR light side-emitting light tube including the IR light side-emitting optical fiber, which includes a core, and an IR light jacket at least partially surrounding the IR light side-emitting optical fiber, where the IR light jacket is made from a light-transmissive material and has a durometer of at least 50 A and an outer diameter at least seven times a core diameter of the IR light side-emitting optical fiber. The IR light side-emitting optical fiber can include a cladding surrounding the core, which is made from polymethyl methacrylate (PMMA). The red light side-emitting optical fiber can include cladding surrounding a PMMA core. The IR light source can be configured to emit IR light having a peak wavelength between 828 nm and 832 nm at an operating temperature between −10 degrees and 60 degrees C.

Alternatively, for the light therapy wearable in the first paragraph of this section, the red light source can be a first red light source, and the light therapy wearable can further include a second red light source, where the first red light source projects light into a first end of the red light side-emitting optical fiber and the second red light source projects light into a second end of the red light side-emitting optical fiber, where the second end being opposite the first end. In this alternative, the fabric panel can be a first fabric panel, and the light therapy wearable can further include a second fabric panel sewn or otherwise affixed to the first fabric panel, where the first red light source is mounted to the first fabric panel and the second red light source is mounted to the second fabric panel.

Alternatively, for the light therapy wearable in the first paragraph of this section, the fabric panel can be a first fabric panel, and the light therapy wearable can further include a second fabric panel sewn or otherwise affixed to the first fabric panel, where the IR light side-emitting optical fiber is a first IR light side-emitting optical fiber, and the light therapy wearable further includes a second IR light side-emitting optical fiber, and where the first IR light side-emitting optical fiber is affixed to or held against the first fabric panel and the second IR light side-emitting optical fiber is affixed to or held against the second fabric panel. In this alternative, the IR light source can be a first IR light source, and the light therapy wearable can further include a second IR light source, where the first IR light source is mounted to the first fabric panel and the second IR light source is mounted to the second fabric panel. Also for this alternative, the first IR light side-emitting optical fiber has two ends and the first IR light source projects light into each end of the two ends of the first IR light side-emitting optical fiber, and the second IR light side-emitting optical fiber has two ends and the second IR light source projects light into each end of the two ends of the second IR light side-emitting optical fiber.

Alternatively, for the light therapy wearable in the first paragraph of this section, the fabric panel can be a first fabric panel, and the light therapy wearable can further include a second fabric panel sewn or otherwise affixed to the first fabric panel, where the light therapy wearable further includes a first light pod including a first light pod housing mountable to the first fabric panel and a second light pod including a second light pod housing mountable to the second fabric panel. For this alternative, the red light source can be a first red light source, and the light therapy wearable can further include a second red light source, where the first red light source projects light into a first end of the red light side-emitting optical fiber and the second red light source projects light into a second end of the red light side-emitting optical fiber, where the second end being opposite the first end, and the IR light side-emitting optical fiber is a first IR light side-emitting optical fiber, and the light therapy wearable further includes a second IR light side-emitting optical fiber, where the first IR light side-emitting optical fiber is affixed to or held against the first fabric panel and the second IR light side-emitting optical fiber is affixed to or held against the second fabric panel, and the first red light source and the first IR light source are disposed in the first light pod housing and the second red light source and the second IR light source are disposed in the second light pod housing. For this alternative, the red light source can be a first red light source, and the light therapy wearable further includes a second red light source, where the first red light source projects light into a first end of the red light side-emitting optical fiber and the second red light source projects light into a second end of the red light side-emitting optical fiber, where the second end being opposite the first end, and the IR light side-emitting optical fiber is a first IR light side-emitting optical fiber, and the light therapy wearable further includes a second IR light side-emitting optical fiber, where the first IR light side-emitting optical fiber has two ends and the first IR light source projects light into each end of the two ends of the first IR light side-emitting optical fiber, and the second IR light side-emitting optical fiber has two ends and the second IR light source projects light into each end of the two ends of the second IR light side-emitting optical fiber, where the first red light source and the first IR light source are disposed in the first light pod housing and the second red light source and the second IR light source are disposed in the second light pod housing.

The light therapy wearable in the first paragraph of this section can further include a red light side-emitting light tube including the red light side-emitting optical fiber, which includes a core, and a red light jacket at least partially surrounding the red light side-emitting optical fiber, where the red light jacket is made from a light-transmissive material and has a durometer of at least 50 A and an outer diameter at least seven times a core diameter of the red light side-emitting optical fiber. For this alternative, the red light side-emitting optical fiber includes a cladding surrounding the core.

The light therapy wearable in the first paragraph of this section can further include an IR light side-emitting light tube including the IR light side-emitting optical fiber, which includes a core, and an IR light jacket at least partially surrounding the IR light side-emitting optical fiber, where the IR light jacket is made from a light-transmissive material and having a durometer of at least 50 A and an outer diameter at least seven times a core diameter of the IR light side-emitting optical fiber. For this alternative, the IR light side-emitting optical fiber includes cladding surrounding the core.

The IR light side-emitting optical fiber for the light therapy wearable in the first paragraph of this section can be configured to emit IR light having a peak wavelength between 828 nm and 832 nm at an operating temperature between −10 degrees and 60 degrees C.

The core of the IR light side-emitting optical fiber for the light therapy wearable in the first paragraph of this section can be made from polymethyl methacrylate (PMMA).

In an alternative arrangement, a light therapy wearable can include a fabric panel, at least two side-emitting optical fibers affixed to or held against the fabric panel; a red light source and an infrared (IR) light source. The red light source can be configured to emit light having a wavelength between 630 nm and 700 mm and be optically connected with a red light side-emitting optical fiber of the at least two side-emitting optical fibers. The IR light source can be configured to emit IR light having a wavelength up to 850 nm, and be optically connected with an IR light side-emitting optical fiber of the at least two side-emitting optical fibers. The wavelength of light from the IR light source has a spectral attenuation in the IR light side-emitting optical fiber no more than 7 times that of a spectral attenuation of light from the red light source in the red light side-emitting optical fiber.

### BRIEF DESCRIPTION OF THE DRAWINGS

FIG. 1 is a front view of a light therapy garment in the form of joggers.

FIG. 2 is a left side view of the joggers of FIG. 1.

FIG. 3 is a right side view of the joggers of FIG. 1.

FIG. 4 is a plan view of panels that make up the joggers in FIG. 1 prior to being assembled together.

FIG. 5 is a schematic view of a light pod for use with the joggers of FIG. 1.

FIG. 6 is a cross-sectional view a side-emitting optical fiber for the joggers of FIG. 1.

FIG. 7 is a cross-sectional view a side-emitting light tube for the joggers of FIG. 1.

FIG. 8 is a cross-sectional view of the side-emitting light tube held against an inner surface of a panel.

FIG. 9 is another cross-sectional view of the side-emitting light tube held against the inner surface of the panel.

### DETAILED DESCRIPTION

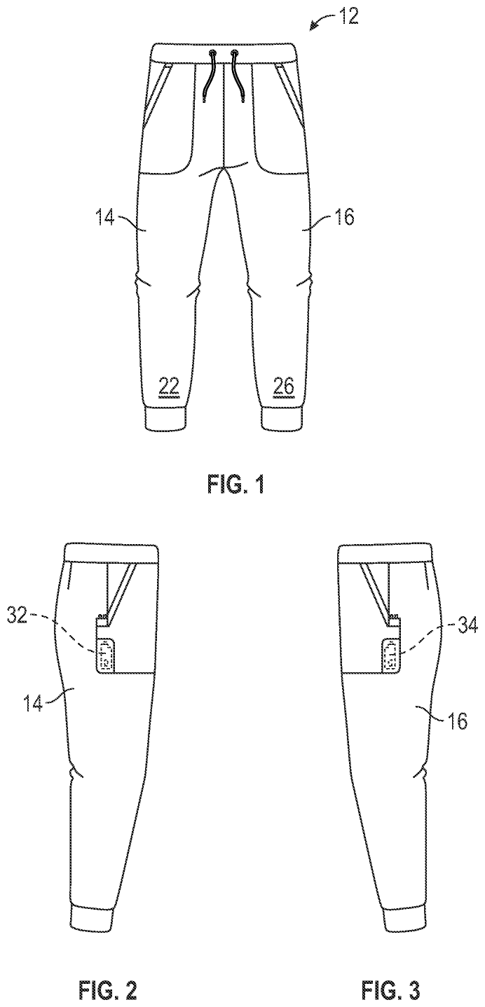

FIGS. 1-3 depict a light therapy wearable 12 in the form joggers. The light therapy wearable 12 is manufactured in a manner so as to project light having a therapeutic wavelength toward a person wearing the wearable. The light therapy wearable 12 can be configured to project light toward targeted body areas, which can include particular muscles, muscle groups, joints, human extremities, and the wearer's skin as examples. The light therapy wearable 12 shown in FIGS. 1-3 is only one example, and the light therapy wearable can be other types of garments, such as other types of tops including hoodies and long-sleeve shirts, and other types of bottoms including shorts and longer pants. When the light therapy wearable 12 is in the form of a garment, the light therapy wearable 12 can be designed to be worn by a person in a similar manner as a conventional garment, so that the joggers shown in FIGS. 1-3 are worn over the legs of a person. The light therapy wearable 12 can also be in the form of a wrap configured to be wrapped around a wearer's arm or leg, for example.

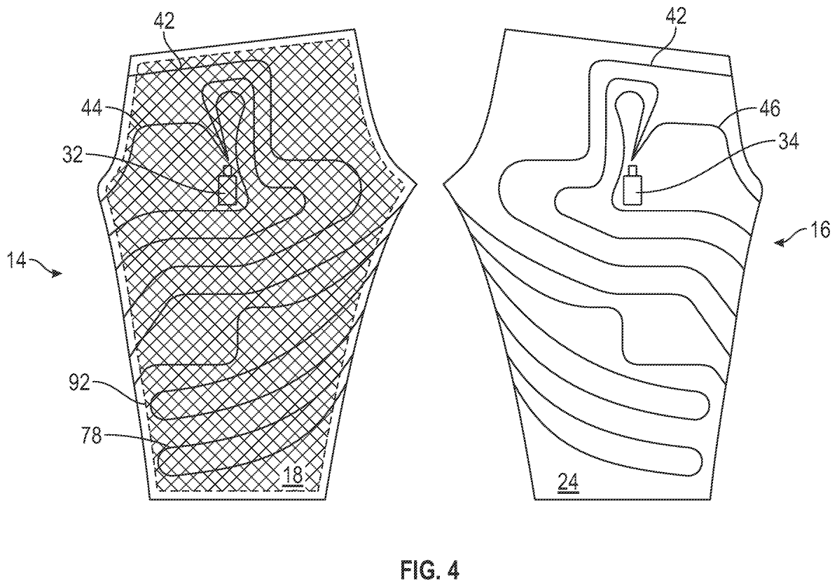

With reference to FIGS. 1-3, the light therapy wearable 12 includes a fabric panel: a first fabric panel 14 and a second fabric panel 16 are depicted in FIGS. 1 and 2, but a greater or fewer number may be provided depending on the form of the light therapy wearable 12. For example, the light therapy wearable 12 shown in FIGS. 1-3 includes cuffs and a waistband that are not depicted in FIG. 4. With particular reference to FIG. 2, the first fabric panel 14 includes a first inner surface 18 that when the light therapy wearable 12 is worn is configured to face a wearer's skin and a first outer surface 22 (see FIG. 1) opposite the first inner surface 18. Similarly, the second fabric panel 16 includes a second inner surface 24 (see FIG. 2) that when the light therapy wearable 12 is worn is configured to face a wearer's skin and a second outer surface 26 (see FIG. 1) opposite the second inner surface 24. For the light therapy wearable 12 depicted in FIGS. 1 and 2, the first outer surface 22 and the second outer surface 26 make up an outermost surface of the light therapy wearable 12, i.e., the surface farthest from the wearer's skin. However, the light therapy wearable 12 may be made up of multiple fabric layers such that the innermost surface may be on one (inner) fabric panel and the outermost surface may be on another (outer) fabric panel. The first fabric panel 14 and the second fabric panel 16 can be either cut and sew pattern pieces or fully-fashioned knitted structures. Examples of yarn from which the fabric panels can be made include cotton, polyester, cotton/polyester blends, microdenier polyester/cotton blends, and combinations thereof.

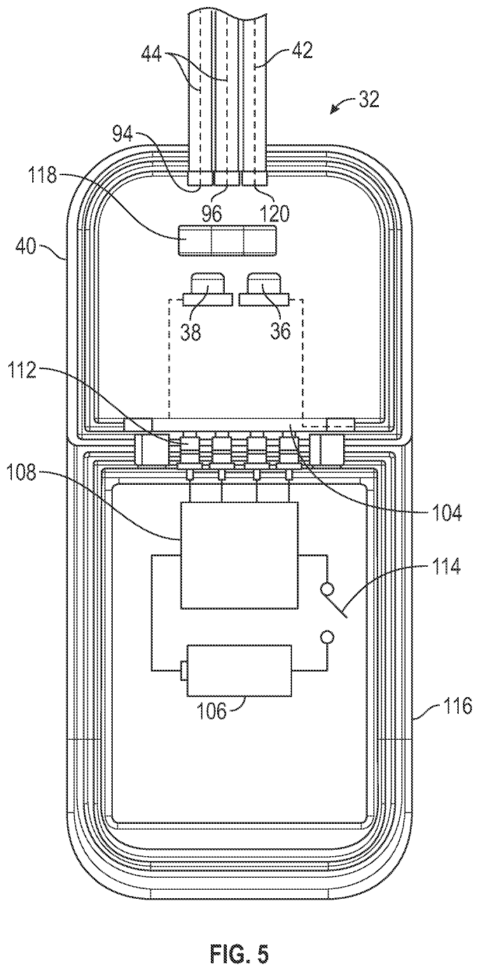

With reference to FIGS. 2-4, optical fiber light source assemblies, which can also be referred to as a first light pod 32 and a second light pod 34 with respect to the illustrated embodiment, are provided on the light therapy wearable 12. For the light therapy wearable 12 depicted in FIGS. 1-4, the first light pod 32 mounts to the first fabric panel 14 and the second light pod 34 mounts to the second fabric panel 14. FIG. 5 schematically depicts the first light pod 32 in more detail, and the second light pod 34 can be identical in configuration. FIG. 5 is a schematic depiction of the first light pod 32 in which an optical fiber light source, which in the illustrated embodiment includes a red laser diode 36 that emits red light and an infrared (IR) laser diode 38 that emits IR radiation, are both received in a light pod housing 40. For example, the red laser diode 36 is configured to emit light having a wavelength of between 630 and 700 nm, and can be more particularly about 660 nm. The IR laser diode 38 is configured to emit IR light having a wavelength less than 850 nm, and more particularly having a peak wavelength between 828 nm and 832 nm at an operating temperature between −10 degrees and 60 degrees C. Other types of light sources, e.g., an LED, could be used instead of the laser diodes. However, no matter whether the IR light source is a laser diode or an LED, the IR light source is configured to emit light having a wavelength less than 850 nm, the advantages of which being described in more detail below.

With reference back to FIG. 4, the light therapy wearable 12 includes at least two side-emitting optical fibers affixed to or held against the fabric panel. More particular to the embodiment depicted in FIG. 4, the light therapy wearable 12 includes a red light side-emitting optical fiber 42, a first IR light side-emitting optical fiber 44 and a second IR light side-emitting optical fiber 46. The red light side-emitting optical fiber 42 is configured to optically connect with a red light source, which in the illustrated embodiment is the red laser diode 36 (FIG. 5) in the first light pod 32 and another red laser diode (not depicted) in the second light pod 34, which can be identical in construction to the first light pod 32. The first IR light side-emitting optical fiber 44 is configured to optically connect with an IR light source, which in the illustrated embodiment is the IR laser diode 38 in the first light pod 32. Different than the red light side-emitting optical fiber 42, in the illustrated embodiment, each of the two opposite ends of the first IR light side-emitting optical fiber 44 optically connects with the IR laser diode 38 in the first light pod 32. Each of the two opposite ends of the second IR light side-emitting optical fiber 46 optically connects with the IR laser diode (not shown) in the second light pod 34, which can be identical in construction to the first light pod 32.

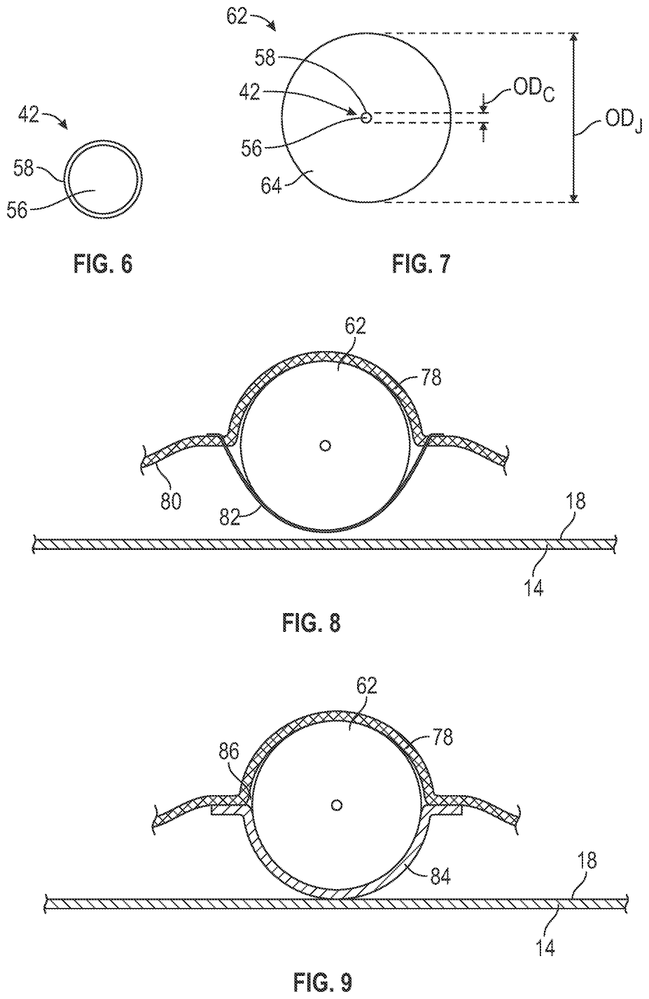

Each of the red light side-emitting optical fiber 42, the first IR light side-emitting optical fiber 44 and the second IR light side-emitting optical fiber 46 is configured to project radiation along the length of and outwardly through its side. Each of the red light side-emitting optical fiber 42, the first IR light side-emitting optical fiber 44 and the second IR light side-emitting optical fiber 46 can be identical in construction so that only a cross section of the red light side-emitting optical fiber 42 is shown in FIG. 6 with the understanding that the first IR light side-emitting optical fiber 44 and the second IR light side-emitting optical fiber 46 can be identically constructed. With reference to FIG. 6, the red light side-emitting optical fiber 42 includes a core 56 and cladding 58 surrounding the core 56. The core 56 in the illustrated embodiment is made from a synthetic polymeric material, e.g., a high-purity polymethyl methacrylate (PMMA). The cladding 58 in the illustrated embodiment is made from a fluorinated polymer. Unlike typical optical fibers, for example an optical fiber used to transmit light between two ends of the optical fiber in fiber-optic communications, the red light side-emitting optical fiber 42 (and the first IR light side-emitting optical fiber 44 and the second IR light side-emitting optical fiber 46) transmits light outwardly along the length of the red light side-emitting optical fiber 42. For example, the cladding 58 can be etched, either mechanically or chemically, to allow light to escape outwardly through the side of the red light side-emitting optical fiber 42 along the length of the red light side-emitting optical fiber 42.

FIG. 7 is a cross-sectional view of a red light side-emitting light tube 62, which could be affixed to or held against the first fabric panel 14 and the second fabric panel 16. The first IR light side-emitting optical fiber 44 and the second IR light side-emitting optical fiber 46 could be provided in a side-emitting light tube having a similar construction, so for the sake of brevity only the red light side-emitting light tube 62 will be described with particularity. The red light side-emitting light tube 62 includes the red light side-emitting optical fiber 42 and a jacket 64 at least partially surrounding the red light side-emitting optical fiber 42. The jacket 64 in the illustrated embodiment is made from silicone, which can be transparent or translucent, having a durometer of at least 50 A, and preferably at least 65 A. The jacket 64 can be extruded over the red light side-emitting optical fiber 42 such that the jacket 64 surrounds the red light side-emitting optical fiber 42 and is in contact with the cladding 58. Even though the jacket 64 is in contact with the cladding 58 so that no or a very small air gap is provided between them, the red light side-emitting optical fiber 42 is movable with respect to the jacket 64 in an axial direction, which is parallel with a longest dimension of the red light side-emitting light tube 62. Allowing for movement of the red light side-emitting optical fiber 42 with respect to the jacket 64 can be helpful in preventing damage to the red light side-emitting optical fiber 42 when the red light side-emitting light tube 62 is bent.

The outer diameter (OM) of the jacket 64 is at least seven times the core diameter (OD_{c}) of the red light side-emitting optical fiber 42. As an example, the core diameter (OD_{c}) of the red light side-emitting optical fiber 42 can be an average of 0.24 mm (minimum 0.225 mm and maximum 0.255 mm) and the outer diameter (OM) of the jacket 64 between 1.75 mm and 2.4 mm. The red light side-emitting optical fiber 42 could have a smaller core diameter (OD_{c}). As another example, the core diameter (OD_{c}) of the red light side-emitting optical fiber 42 can be 0.5 mm with the outer diameter (OM) of the jacket 64 being between 7 times to 11.5 times greater than the core diameter (OD_{c}) of the red light side-emitting optical fiber 42. Having the core diameter (OD_{c}) of the red light side-emitting optical fiber 42 larger than 0.5 mm with the relatively larger jacket 64 can result in the red light side-emitting light tube 62 being too large in cross-section making it difficult to incorporate into the garment in an aesthetic and practically functional manner. Providing the jacket 64 with the outer diameter (OM) that is between 7 times to 11.5 times greater that the core diameter (OD_{c}) of the red light side-emitting optical fiber 42 protects the red light side-emitting optical fiber 42 against excessive force and dropping. It is also beneficial to inhibit bending the red light side-emitting optical fiber 42 in too tight of an arc, but the jacket 64 still allows needed flexibility to the red light side-emitting light tube 62 and is comfortable against the wearer's skin.

As mentioned above, the side-emitting optical fibers 42, 44 and 46 are affixed to or held against a respective fabric panel. In FIG. 4, the side-emitting optical fibers 42 and 44 are shown held against the first inner surface 18 of the first fabric panel 14 by way of a light-transmissive inner layer 78 affixed to the first fabric panel 14 and at least partially covering the side-emitting optical fibers 42 and 44. The side-emitting optical fibers 42 and 46 can be held against the second inner surface 24 of the second fabric panel 14 by way of a similar light-transmissive inner layer (not shown) affixed to the second fabric panel 14 in a similar manner and at least partially covering the side-emitting optical fibers 42 and 46.

Where the side-emitting optical fibers 42, 44 and 46 are provided in a jacket, similar to the jacket 64 in FIG. 7, the respective side-emitting optical light tubes could be held against the first inner surface 18 of the first fabric panel 14 and the second inner surface 24 of the second fabric panel 14 by way of a similar light-transmissive inner layer. For example, FIG. 8 shows the red light side-emitting light tube 62 is held against an outer surface 80, which faces away from a wearer's skin, of the light-transmissive inner layer 78 through thread stitches 82, which can be embroidery or more typical sewing stitches. The light-transmissive inner layer 78 is affixed to the first fabric panel 14 in a manner that is described in more detail below. The manner in which the light-transmissive inner layer 78 is affixed to the first fabric panel 14 allows for movement of the red light side-emitting light tube 62 and portions of the light-transmissive inner layer 78 with respect to the first fabric panel 14. This allows for the wearer of the light therapy wearable 12 (or other similarly constructed wearable) to move freely while wearing the light therapy wearable 12 with the red light side-emitting light tube 62 being maintained adjacent to the wearer's skin. Also, the stitches 82 may allow for movement of the red light side-emitting light tube 62 with respect to the light-transmissive inner layer 78 and the first fabric panel 14 in an axial direction, which is parallel with the longest dimension of the red light side-emitting light tube 62.

As seen in FIG. 9, in lieu of the stitches 82, an intermediate channel layer 84 can be positioned between the first fabric panel 14 and the light-transmissive inner layer 78. The intermediate channel layer 84 can be affixed to the light-transmissive inner layer 78 to define a channel 86 in which the red light side-emitting light tube 62 is received. In the embodiment illustrated in FIGS. 4, 8 and 9, the light-transmissive inner layer 78 is a fabric mesh material. Examples of such fabric mesh material include sports mesh, power mesh, mesh netting, tulle, nylon mesh, and polyester mesh; however, other light-transmissive materials could be employed. The intermediate channel layer 84 can be any material composition such as cotton, polyester, spandex affixed to the light-transmissive inner layer 78. For example, the intermediate channel layer 84 can be a jersey cotton fabric sewn to the light-transmissive inner layer 78. Alternatively, the intermediate channel layer 84 can be a jersey cotton bias tape that is adhered, bonded or sewn to the light-transmissive inner layer 78. The intermediate channel layer 84 can also be reflective of red and infrared light, while still being comfortable. For example, the intermediate channel layer 84 can be a white or red dyed jersey cotton fabric, which will reflect red light impinging on the intermediate channel layer 84 back towards the wearer. Interestingly, it was discovered that a red dyed jersey cotton fabric intermediate channel layer 84 worked better for reflection as compared to a red print sublimated jersey cotton fabric intermediate channel layer 84.

Affixing the intermediate channel layer 84 to the light-transmissive inner layer 78 instead of to the first fabric panel 14 will not result in a seam noticeable on the exterior of the light therapy wearable 12 allowing the therapeutic wearable to look from the exterior more similar to a typical garment that does not include side-emitting optical fibers. As mentioned above, the light-transmissive inner layer 78 can at least partially cover the red light side-emitting light tube 62 and/or the light-transmissive inner layer 78 can cover nearly the entirety of the first inner surface 18 of the first fabric panel 14. For example, the light-transmissive inner layer 78 can cover the entirety of the first inner surface 18 within the area bounded by the broken line 92 shown in FIG. 4, which is provided to allow the first fabric panel 14 to be sewn to the second fabric panel 14 as well as to allow for hemming, which is typical in a conventional garment. Additionally, the light-transmissive inner layer 78 and the intermediate channel layer 84 can be employed with IR light side-emitting light tubes (constructed similarly to the red light side-emitting light tube 62) and with the second fabric panel 14 in a similar manner.

The light-transmissive inner layer 78 can be sewn to the first fabric panel 14 along the broken line 92 shown in FIG. 2 and allow for the light-transmissive inner layer 78 to move away from the first inner surface 18 of the first fabric panel 14 at locations offset from where they are sewn together. This allows for movement of the red light side-emitting light tube 62 with respect to the first fabric panel 14 in both an axial direction, which is parallel with the longest dimension of the red light side-emitting light tube 62, and a transverse direction, which is perpendicular to the axial direction. As illustrated, the light-transmissive inner layer 78 is affixed to the first fabric panel 14 offset from where the intermediate channel layer 84 is affixed to the light-transmissive inner layer 78 so that the light-transmissive inner layer 78 is movable in three mutually perpendicular axes with respect to the first fabric panel 14 along at least a portion of the channel 86.

As light travels along a length of optical fiber, some light is absorbed and some light is scattered, i.e., emitted through the side of the optical fiber. The sum of these absorption and scattering losses is referred to as attenuation. For side-emitting optical fiber, this attenuation can be used to determine what percentage of light remains in the optical fiber at a particular length along the optical fiber. Using long lengths of side-emitting optical fiber allows more light to escape out the sides of the optical fiber and towards the skin of the wearer of the wearable. Conversely, using short lengths of side-emitting optical fiber allows for very consistent lighting from the sides of the optical fiber; however, this comes along with less light exiting the sides of the optical fiber. Additionally, the attenuation of the side-emitting optical fiber is also a function of the wavelength of light travelling through the fiber. These factors were balanced to provide an appropriate length the red light side-emitting optical fiber 42 and each IR light side-emitting optical fiber 44, 46. So that the red light side-emitting optical fiber 42 is not considerably longer than each IR light side-emitting optical fiber 44, 46, the wavelength of light from the IR light source, e.g., the IR laser diode 38, can have a spectral attenuation in the IR light side-emitting optical fibers 44, 46 no more than 7 times that of a spectral attenuation of light from the red light source, e.g., the red laser diode 36, in the red light side-emitting optical fiber 42.

With reference to FIG. 4 and when each side-emitting optical fiber 42, 44 and 46 includes the PMMA core 56 and fluorinated polymer cladding 58, which is commercially available from Toray Industries, Inc. under the brand Toray Raytella side light plastic optical fiber “PS series,” the red light side-emitting optical fiber 42 is at least 1.95 times longer than each IR light side-emitting optical fiber 44, 46. For the embodiment shown in FIG. 4, the red light side-emitting optical fiber 42 is 5.76 m, and each IR light side-emitting optical fiber 44, 46 is 2.91 m. Surprisingly, the attenuation for 850 nm IR light through the IR light side-emitting optical fibers 44 and 46 is about 5.47 db/m, but the attenuation for 830 nm IR light through the side-emitting optical fiber 42, 44 and 46 is about 2.98 db/m. If 850 nm IR light were directed into the IR light side-emitting optical fibers 44 and 46, then the IR light side-emitting optical fibers 44 and 46 would need to be much shorter to have at least about 50% of the maximum light output at a location near the middle of the length of each IR light side-emitting optical fiber 44 and 46. By directing 830 nm IR light into each of the two ends of each IR light side-emitting optical fiber 44 and 46, the benefits of IR light therapy can be achieved while using a longer fiber.

With reference back to FIG. 5, the first light pod 32 is shown in more detail, and the second light pod 34 can be identical in configuration. A light source electrical contact 104, which can include electrical receptacles and appropriate circuitry (not depicted) is in electrical communication (depicted schematically) with the red laser diode 36 and the IR laser diode 38. A power source, such as a battery 106 is in electrical communication with a controller 108, which is electrically connected with power source electrical contacts 112. The controller 108 can be an appropriate integrated circuit, for example. The controller 108 is in electrical communication with a switch 114 that can control power delivery to the controller 108, the red laser diode 36 and the IR laser diode 38. The battery 106, the controller 108 and the switch 114 can be received in a power source housing 116, and an actuator (not shown), e.g., a button, can be accessible to a user on the outside of the power source housing 116 for operating the switch 114. The power source electrical contacts 112, which can be pogo pins, can connect with the light source electrical contact 104 when the power source housing 116 is connected with the light pod housing 40.

An optic (or optics) 118 is provided in the light pod housing 40 and cooperates with the red laser diode 36 and with the IR laser diode 38. The optic 118 directs the light emanating from the red laser diode 36 into a first end 120 of the red light side-emitting optical fiber 42. The optic 118 directs the light emanating from the IR laser diode 38 into each end, i.e., a first end 94 and a second, opposite, end 96 of the first IR side-emitting optical fiber 44. As mentioned above, the second light pod 34 can be identical in configuration to the first light pod 32. As such, the second, opposite, end (see FIG. 4, but no reference number is provided because of the size) of the red light side-emitting optical fiber 42 connects with the second light pod 34. Similarly, each end of the second IR side-emitting optical fiber 46 can optically connect with the second light pod 34.

It will be appreciated that various of the above-disclosed embodiments and other features and functions, or alternatives or varieties thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. A light therapy wearable comprising:
a fabric panel;
at least two side-emitting optical fibers affixed to or held against the fabric panel;
a red light source configured to emit light having a wavelength between 630 and 700 nm and being optically connected with a red light side-emitting optical fiber of the at least two side-emitting optical fibers; and
an infrared (IR) light source configured to emit IR light having a wavelength less than 850 nm, the IR light source being optically connected with an IR light side-emitting optical fiber of the at least two side-emitting optical fibers, wherein the IR light side-emitting optical fiber includes a core being made from a synthetic polymeric material.

2. The light therapy wearable of claim 1, wherein the wavelength of light from the IR light source has a spectral attenuation in the IR light side-emitting optical fiber no more than 7 times that of a spectral attenuation of light from the red light source in the red light side-emitting optical fiber.

3. The light therapy wearable of claim 2, wherein the red light source is a first red light source, and the light therapy wearable further includes a second red light source, wherein the first red light source projects light into a first end of the red light side-emitting optical fiber and the second red light source projects light into a second end of the red light side-emitting optical fiber, the second end being opposite the first end.

4. The light therapy wearable of claim 3, wherein the fabric panel is a first fabric panel, and the light therapy wearable further includes a second fabric panel sewn or otherwise affixed to the first fabric panel, wherein the first red light source is mounted to the first fabric panel and the second red light source is mounted to the second fabric panel.

5. The light therapy wearable of claim 4, wherein the IR light side-emitting optical fiber is a first IR light side-emitting optical fiber, and the light therapy wearable further includes a second IR light side-emitting optical fiber, wherein the first IR light side-emitting optical fiber is affixed to or held against the first fabric panel and the second IR light side-emitting optical fiber is affixed to or held against the second fabric panel.

6. The light therapy wearable of claim 5, wherein the IR light source is a first IR light source, and the light therapy wearable further includes a second IR light source, wherein the first IR light source is mounted to the first fabric panel and the second IR light source is mounted to the second fabric panel.

7. The light therapy wearable of claim 6, wherein the first IR light side-emitting optical fiber has two ends and the first IR light source projects light into each end of the two ends of the first IR light side-emitting optical fiber, and the second IR light side-emitting optical fiber has two ends and the second IR light source projects light into each end of the two ends of the second IR light side-emitting optical fiber.

8. The light therapy wearable of claim 7, further comprising a first light pod including a first light pod housing mountable to the first fabric panel and a second light pod including a second light pod housing mountable to the second fabric panel, wherein the first red light source and the first IR light source are disposed in the first light pod housing and the second red light source and the second IR light source are disposed in the second light pod housing.

9. The light therapy wearable of claim 8, further comprising a red light side-emitting light tube including the red light side-emitting optical fiber, and a red light jacket at least partially surrounding the red light side-emitting optical fiber, the red light jacket being made from a light-transmissive material and having a durometer of at least 50 A and an outer diameter at least seven times a core diameter of the red light side-emitting optical fiber.

10. The light therapy wearable of claim 9, further comprising an IR light side-emitting light tube including the IR light side-emitting optical fiber, and an IR light jacket at least partially surrounding the IR light side-emitting optical fiber, the IR light jacket being made from a light-transmissive material and having a durometer of at least 50 A and an outer diameter at least seven times a core diameter of the IR light side-emitting optical fiber.

11. The light therapy wearable of claim 10, wherein the IR light side-emitting optical fiber includes cladding surrounding the core, which is made from polymethyl methacrylate (PMMA).

12. The light therapy wearable of claim 11, wherein the red light side-emitting optical fiber includes cladding surrounding a PMMA core.

13. The light therapy wearable of claim 12, wherein the IR light source is configured to emit IR light having a peak wavelength between 828 nm and 832 nm at an operating temperature between −10 degrees and 60 degrees C.

14. The light therapy wearable of claim 1, wherein the red light source is a first red light source, and the light therapy wearable further includes a second red light source, wherein the first red light source projects light into a first end of the red light side-emitting optical fiber and the second red light source projects light into a second end of the red light side-emitting optical fiber, the second end being opposite the first end.

15. The light therapy wearable of claim 14, wherein the fabric panel is a first fabric panel, and the light therapy wearable further includes a second fabric panel sewn or otherwise affixed to the first fabric panel, wherein the first red light source is mounted to the first fabric panel and the second red light source is mounted to the second fabric panel.

16. The light therapy wearable of claim 1, wherein the fabric panel is a first fabric panel, and the light therapy wearable further includes a second fabric panel sewn or otherwise affixed to the first fabric panel,
wherein the IR light side-emitting optical fiber is a first IR light side-emitting optical fiber, and the light therapy wearable further includes a second IR light side-emitting optical fiber, and
wherein the first IR light side-emitting optical fiber is affixed to or held against the first fabric panel and the second IR light side-emitting optical fiber is affixed to or held against the second fabric panel.

17. The light therapy wearable of claim 16, wherein the IR light source is a first IR light source, and the light therapy wearable further includes a second IR light source, wherein the first IR light source is mounted to the first fabric panel and the second IR light source is mounted to the second fabric panel.

18. The light therapy wearable of claim 17, wherein the first IR light side-emitting optical fiber has two ends and the first IR light source projects light into each end of the two ends of the first IR light side-emitting optical fiber, and the second IR light side-emitting optical fiber has two ends and the second IR light source projects light into each end of the two ends of the second IR light side-emitting optical fiber.

19. The light therapy wearable of claim 1, wherein the fabric panel is a first fabric panel, and the light therapy wearable further includes a second fabric panel sewn or otherwise affixed to the first fabric panel,
the light therapy wearable further comprising a first light pod including a first light pod housing mountable to the first fabric panel and a second light pod including a second light pod housing mountable to the second fabric panel.

20. The light therapy wearable of claim 19, wherein the red light source is a first red light source, and the light therapy wearable further includes a second red light source, wherein the first red light source projects light into a first end of the red light side-emitting optical fiber and the second red light source projects light into a second end of the red light side-emitting optical fiber, the second end being opposite the first end,
wherein the IR light side-emitting optical fiber is a first IR light side-emitting optical fiber, and the light therapy wearable further includes a second IR light side-emitting optical fiber, wherein the first IR light side-emitting optical fiber is affixed to or held against the first fabric panel and the second IR light side-emitting optical fiber is affixed to or held against the second fabric panel.

21. The light therapy wearable of claim 19, wherein the red light source is a first red light source, and the light therapy wearable further includes a second red light source, wherein the first red light source projects light into a first end of the red light side-emitting optical fiber and the second red light source projects light into a second end of the red light side-emitting optical fiber, the second end being opposite the first end,
wherein the IR light side-emitting optical fiber is a first IR light side-emitting optical fiber, and the light therapy wearable further includes a second IR light side-emitting optical fiber, wherein the first IR light side-emitting optical fiber has two ends and the first IR light source projects light into each end of the two ends of the first IR light side-emitting optical fiber, and the second IR light side-emitting optical fiber has two ends and the second IR light source projects light into each end of the two ends of the second IR light side-emitting optical fiber; and
wherein the first red light source and the first IR light source are disposed in the first light pod housing and the second red light source and the second IR light source are disposed in the second light pod housing.

22. The light therapy wearable of claim 1, further comprising a red light side-emitting light tube including the red light side-emitting optical fiber, and a red light jacket at least partially surrounding the red light side-emitting optical fiber, which includes a core made from PMMA, the red light jacket being made from a light-transmissive material and having a durometer of at least 50 A and an outer diameter at least seven times a core diameter of the red light side-emitting optical fiber.

23. The light therapy wearable of claim 22, wherein the red light side-emitting optical fiber includes a cladding surrounding the core.

24. The light therapy wearable of claim 1, further comprising an IR light side-emitting light tube including the IR light side-emitting optical fiber, and an IR light jacket at least partially surrounding the IR light side-emitting optical fiber, the IR light jacket being made from a light-transmissive material and having a durometer of at least 50 A and an outer diameter at least seven times a core diameter of the IR light side-emitting optical fiber.

25. The light therapy wearable of claim 24, wherein the IR light side-emitting optical fiber includes cladding surrounding the core.

26. The light therapy wearable of claim 1, wherein the IR light source is configured to emit IR light having a peak wavelength between 828 nm and 832 nm at an operating temperature between −10 degrees and 60 degrees C.

27. A light therapy wearable comprising:
a fabric panel;
at least two side-emitting optical fibers affixed to or held against the fabric panel;
a red light source configured to emit light having a wavelength between 630 nm and 700 mm and being optically connected with a red light side-emitting optical fiber of the at least two side-emitting optical fibers; and
an infrared (IR) light source configured to emit IR light having a wavelength up to 850 nm, the IR light source being optically connected with an IR light side-emitting optical fiber of the at least two side-emitting optical fibers, wherein the wavelength of light from the IR light source has a spectral attenuation in the IR light side-emitting optical fiber no more than 7 times that of a spectral attenuation of light from the red light source in the red light side-emitting optical fiber.

28. The light therapy wearable of claim 27, wherein the red light source is a first red light source, and the light therapy wearable further includes a second red light source, wherein the first red light source projects light into a first end of the red light side-emitting optical fiber and the second red light source projects light into a second end of the red light side-emitting optical fiber, the second end being opposite the first end.

29. The light therapy wearable of claim 28, wherein the fabric panel is a first fabric panel, and the light therapy wearable further includes a second fabric panel sewn or otherwise affixed to the first fabric panel, wherein the first red light source is mounted to the first fabric panel and the second red light source is mounted to the second fabric panel.

30. The light therapy wearable of claim 29, wherein the IR light side-emitting optical fiber is a first IR light side-emitting optical fiber, and the light therapy wearable further includes a second IR light side-emitting optical fiber, wherein the first IR light side-emitting optical fiber is affixed to or held against the first fabric panel and the second IR light side-emitting optical fiber is affixed to or held against the second fabric panel.

31. The light therapy wearable of claim 30, wherein the IR light source is a first IR light source, and the light therapy wearable further includes a second IR light source, wherein the first IR light source is mounted to the first fabric panel and the second IR light source is mounted to the second fabric panel.

32. The light therapy wearable of claim 31, wherein the first IR light side-emitting optical fiber has two ends and the first IR light source projects light into each end of the two ends of the first IR light side-emitting optical fiber, and the second IR light side-emitting optical fiber has two ends and the second IR light source projects light into each end of the two ends of the second IR light side-emitting optical fiber.

33. The light therapy wearable of claim 32, further comprising a first light pod including a first light pod housing mountable to the first fabric panel and a second light pod including a second light pod housing mountable to the second fabric panel, wherein the first red light source and the first IR light source are disposed in the first light pod housing and the second red light source and the second IR light source are disposed in the second light pod housing.

34. The light therapy wearable of claim 33, further comprising a red light side-emitting light tube including the red light side-emitting optical fiber, and a red light jacket at least partially surrounding the red light side-emitting optical fiber, the red light jacket being made from a light-transmissive material and having a durometer of at least 50 A and an outer diameter at least seven times a core diameter of the red light side-emitting optical fiber.

35. The light therapy wearable of claim 34, further comprising an IR light side-emitting light tube including the IR light side-emitting optical fiber, and an IR light jacket at least partially surrounding the IR light side-emitting optical fiber, the IR light jacket being made from a light-transmissive material and having a durometer of at least 50 A and an outer diameter at least seven times a core diameter of the IR light side-emitting optical fiber.

36. The light therapy wearable of claim 35, wherein the IR light side-emitting optical fiber includes cladding surrounding the core, which is made from polymethyl methacrylate (PMMA).

37. The light therapy wearable of claim 36, wherein the red light side-emitting optical fiber includes cladding surrounding a PMMA core.

38. The light therapy wearable of claim 37, wherein the IR light source is configured to emit IR light having a peak wavelength between 828 nm and 832 nm at an operating temperature between −10 degrees and 60 degrees C.

39. The light therapy wearable of claim 27, wherein the red light source is a first red light source, and the light therapy wearable further includes a second red light source, wherein the first red light source projects light into a first end of the red light side-emitting optical fiber and the second red light source projects light into a second end of the red light side-emitting optical fiber, the second end being opposite the first end.

40. The light therapy wearable of claim 39, wherein the fabric panel is a first fabric panel, and the light therapy wearable further includes a second fabric panel sewn or otherwise affixed to the first fabric panel, wherein the first red light source is mounted to the first fabric panel and the second red light source is mounted to the second fabric panel.

41. The light therapy wearable of claim 27, wherein the fabric panel is a first fabric panel, and the light therapy wearable further includes a second fabric panel sewn or otherwise affixed to the first fabric panel,
wherein the IR light side-emitting optical fiber is a first IR light side-emitting optical fiber, and the light therapy wearable further includes a second IR light side-emitting optical fiber, and
wherein the first IR light side-emitting optical fiber is affixed to or held against the first fabric panel and the second IR light side-emitting optical fiber is affixed to or held against the second fabric panel.

42. The light therapy wearable of claim 41, wherein the IR light source is a first IR light source, and the light therapy wearable further includes a second IR light source, wherein the first IR light source is mounted to the first fabric panel and the second IR light source is mounted to the second fabric panel.

43. The light therapy wearable of claim 42, wherein the first IR light side-emitting optical fiber has two ends and the first IR light source projects light into each end of the two ends of the first IR light side-emitting optical fiber, and the second IR light side-emitting optical fiber has two ends and the second IR light source projects light into each end of the two ends of the second IR light side-emitting optical fiber.

44. The light therapy wearable of claim 27, wherein the fabric panel is a first fabric panel, and the light therapy wearable further includes a second fabric panel sewn or otherwise affixed to the first fabric panel,
the light therapy wearable further comprising a first light pod including a first light pod housing mountable to the first fabric panel and a second light pod including a second light pod housing mountable to the second fabric panel.

45. The light therapy wearable of claim 44, wherein the red light source is a first red light source, and the light therapy wearable further includes a second red light source, wherein the first red light source projects light into a first end of the red light side-emitting optical fiber and the second red light source projects light into a second end of the red light side-emitting optical fiber, the second end being opposite the first end,
wherein the IR light side-emitting optical fiber is a first IR light side-emitting optical fiber, and the light therapy wearable further includes a second IR light side-emitting optical fiber, wherein the first IR light side-emitting optical fiber is affixed to or held against the first fabric panel and the second IR light side-emitting optical fiber is affixed to or held against the second fabric panel.

46. The light therapy wearable of claim 45, wherein the red light source is a first red light source, and the light therapy wearable further includes a second red light source, wherein the first red light source projects light into a first end of the red light side-emitting optical fiber and the second red light source projects light into a second end of the red light side-emitting optical fiber, the second end being opposite the first end,
wherein the IR light side-emitting optical fiber is a first IR light side-emitting optical fiber, and the light therapy wearable further includes a second IR light side-emitting optical fiber, wherein the first IR light side-emitting optical fiber has two ends and the first IR light source projects light into each end of the two ends of the first IR light side-emitting optical fiber, and the second IR light side-emitting optical fiber has two ends and the second IR light source projects light into each end of the two ends of the second IR light side-emitting optical fiber; and
wherein the first red light source and the first IR light source are disposed in the first light pod housing and the second red light source and the second IR light source are disposed in the second light pod housing.

47. The light therapy wearable of claim 27, further comprising a red light side-emitting light tube including the red light side-emitting optical fiber, and a red light jacket at least partially surrounding the red light side-emitting optical fiber, which includes a core made from PMMA, the red light jacket being made from a light-transmissive material and having a durometer of at least 50 A and an outer diameter at least seven times a core diameter of the red light side-emitting optical fiber.

48. The light therapy wearable of claim 47, wherein the red light side-emitting optical fiber includes a cladding surrounding the core.

49. The light therapy wearable of claim 27, further comprising an IR light side-emitting light tube including the IR light side-emitting optical fiber, and an IR light jacket at least partially surrounding the IR light side-emitting optical fiber, the IR light jacket being made from a light-transmissive material and having a durometer of at least 50 A and an outer diameter at least seven times a core diameter of the IR light side-emitting optical fiber.

50. The light therapy wearable of claim 49, wherein the IR light side-emitting optical fiber includes cladding surrounding the core.

51. The light therapy wearable of claim 27, wherein the IR light source is configured to emit IR light having a peak wavelength between 828 nm and 832 nm at an operating temperature between −10 degrees and 60 degrees C.
